(19) United States
(12) Plant Patent Application Publication
(10) Pub. No.: US 2002/0188975 P1
(43) Pub. Date: Dec. 12, 2002

(54) Chrysanthemum plant named 'MOZA BN 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/864,914

(22) Filed: May 24, 2001

(51) Int. Cl. A01H 5/00

(52) U.S. Cl. PLT/286

(57) ABSTRACT

A new and distinct Chrysanthemum plant variety characterized by a daisy type inflorescence with many tightly held ray florets, consistent flowering response to short days, blooming consistently after 49 days of short day length, compact growth habit, and consistent natural season flowering habit during mid September.

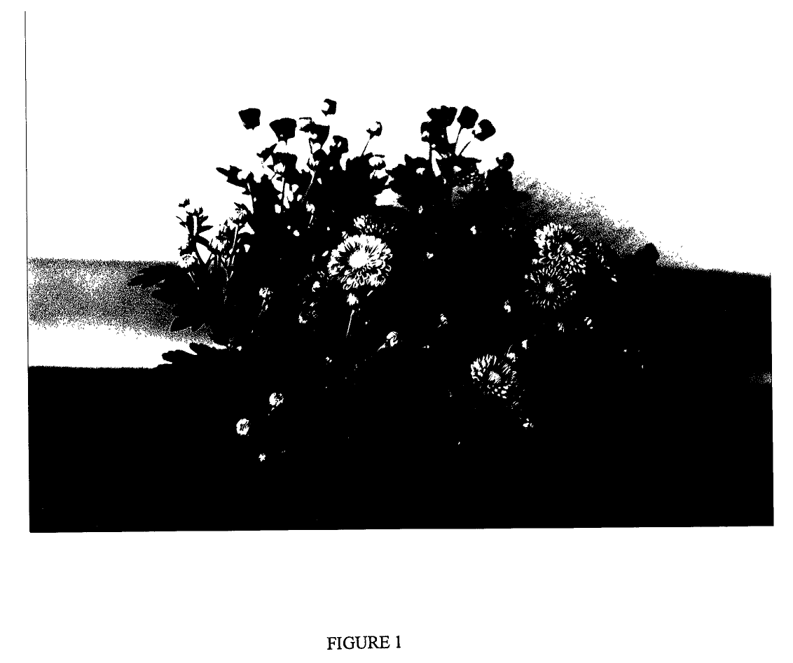

‘MOZA BN 01’: Table of R.H.S. Color Codes

| | | | |
|---|---|---|---|
| FOLIAGE | Leaf | young foliage upper surface | Near 137A |
| | | young foliage under surface | Near 137C |
| | | mature foliage upper surface | Near 137A |
| | | mature foliage under surface | Near 147B |
| | | venation upper surface | Near 146A |
| | | venation under surface | Near 146B |
| BLOOM | Ray Florets | upper surface at first opening, | Near 13B |
| | | base | |
| | | upper surface at first opening, | Near 146A |
| | | apex | |
| | | upper surface at maturity, base | Near 13B |
| | | upper surface at maturity, apex | Near 146A |
| | | upper surface at fading, base | Near 13B |
| | | upper surface at fading, apex | Near 146A |
| | | under surface at first opening, | Near 13B |
| | | base | |
| | | under surface at first opening, | Near 146A |
| | | apex | |
| | | under surface at maturity, base | Near 13B |
| | | under surface at maturity, apex | Near 146A |
| | | under surface at fading, base | Near 13B |
| | | under surface at fading, apex | Near 146A |
| | | Pistillate: Stigma | Near 15A |
| | Disc Florets | at first opening | Near 15A |
| | | at maturity | Near 15A |
| | | at fading | Near 15A |
| | | Staminate: Anther | Near 15A |
| | | Staminate: Pollen | Near 15A |
| | Peduncle | | Near 137C |
| | Inflorescence | | Near 137B |
| | Bud | | |
| | Sepals | upper surface | Near 137C |
| | | under surface | Near 137C |

### BACKGROUND OF THE INVENTION

The present invention relates to a new and distinct cultivar of Chrysanthemum plant, botanically known as Dendranthema morifolium and herein after referred to by the cultivar name ‘MOZA BN 01.’

This new cultivar originated from hybridization of a female parent Chrysanthemum cultivar (unpatented in the United States) with a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, CA and has shown that the unique features of this new cultivar are stable and reproduced true to type on successive generations.

### SUMMARY OF THE INVENTION

The cultivar ‘MOZA BN 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA BN 01.’ These characteristics in combination distinguish ‘MOZA BN 01’ as a new and distinct Chrysanthemum:

1. Daisy type inflorescence with many tightly held ray florets;

2. Consistent flowering response to short days, blooming consistently after 49 days of short day length;

3. Compact growth habit; and

4. Consistent natural season flowering habit during mid September.

Plants of the new cultivar are similar to plants of the female parent cultivar in most horticultural characteristics, however plants of the new cultivar differed from plants of the female parent cultivar in the following characteristics:

1. The new cultivar has different color ray florets;

2. The new cultivar is shorter;

3. The new cultivar has shorter internode length; and

4. The new cultivar has many more ray florets than the parent.

Plants of the new cultivar are similar to plants of the male parent cultivar in most horticultural characteristics, however plants of the new cultivar differed from plants of the male parent cultivar in the following characteristics:

1. The new cultivar has different color ray florets;

2. The new cultivar is taller; and

3. The new cultivar has a different type bloom.

### BRIEF DESCRIPTION OF THE PHOTOGRAPHS

The photograph shows a plant of the new cultivar grown in a 4-inch container. A single cutting was propagated in the container. It was planted in mid-July and grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘MOZA BN 01’

Commercial classification: Garden-type Chrysanthemum

Parentage: Hybridization of female parent Chrysanthemum cultivar (unpatented in the United States) with a male parent Chrysanthemum cultivar (unpatented in the United States)

Propagation

Time to rooting.—7 to 14 days at approximately 21° C.

Rooting habit.—Fine, fibrous

Plant

Growth habit.—Mounding herbaceous perennial

Height.—Approximately 19 cm

Growth rate.—Moderate

Branching characteristics.—Free Branching

Length of lateral branches.—Approximately 15 cm

Number of leaves per lateral branch.—Approximately 12

Stem Color.—Near 137B

Foliage

Leaf.—Arrangement: Alternate single Average Length: Approximately 4 cm Average Width: Approximately 2.4 cm Shape of blade: Ovate Apex: Acuminate Base: Attenuate Attachment: Stalked Margin: Serrate Texture of top surface: Lightly pubescent Texture of bottom surface: Lightly pubescent Color: young foliage upper side: Near 137A young foliage under side: Near 137C mature foliage upper side: Near 137A mature foliage under side. Near 137C venation upper side: Near 146A venation under side: Near 146B

Petiole.—Average Length: Approximately 1.5 cm

Bloom

Inflorescence.—Flowering habit: Induced by darkness period greater than 13.5 hours, approximately 49 days of appropriate day length required to induce and develop blooms In lorescence form: Daisy Natural flowering season: Approximately mid-September Number of inflorescences per stem: Approximately 15 to 20 Inflorescence diameter: Approximately 3.2 cm Inflorescence depth: Approximately 1.3 cm Disc diameter: Approximately 1 cm Inflorescence longevity on plant: Approximately 26-29 days Persistence: Persistent

Ray florets.—Appearance: Matte Texture: Smooth Average Number per flower: Approximately 60 to 70 Shape: Cylindric Margin: Entire Apex: Obtuse Length: Approximately 1.1 cm Color: upper surface at first opening: on 20% nearest base: Near 13B upper surface at frst opening: on 80% nearest apex: Near 146A upper surface at maturity: on 30% nearest base: Near 13B upper surface at maturity: on 70% nearest apex: Near 146A upper surface at fading: on 40% nearest base: Near 13B upper surface at fading: on 60% nearest apex: Near 146A under surface at first opening: on 20% nearest base: Near 13B under surface at first opening: on 80% nearest apex: Near 146A under surface at maturity: on 30% nearest base: Near 13B under surface at maturity: on 70% nearest apex: Near 146A under surface at fading: on 40% nearest base: Near 13B under surface atfading: on 60% nearest apex: Near 146A

Disc florets.—Appearance: Shiny Texture: Smooth Average numberperflower: Approximately 80 to 90 Shape: Cylindric Apex.- Obtuse Average Length: Approximately 0.4 cm Average Width: Approximately 0.1 cm Color: at first opening. Near 15A at maturity: Near 15A at fading: Near 15A

Peduncle.—Length: at terminal end (shortest): Approximately 2.2 cm at lateral end longest): Approximately 4.3 cm Strength: Moderate Color: Near 137C Habit: Erect

Inflorescence bud.—Length: Approximately 0.4 cm Diameter: Approximately 0.5 cm Form: Globular Color: Near 137B

Sepals.—Appearance: Matte Texture: Lightly pubescent Number: Approximately 20 to 30 Shape: Ovate Margin: Entire Apex: Acute Color: upper side: Near 137C under side: Near 137C

Reproductive Organs

Ray florets.—Pistillate: Number ofpistils per flower: 1 Stigma shape: 2 branched Stigma color: Near 15A Stigma Length: Approximately 0.1 cm Style length: Approximately 0.3 cm

Disc florets.—Pistillate: Number ofpistils per flower: 1 Stigma shape: Cylindric Staminate: Number ofstamens per flower: Approximately 5 Anther shape: Tubular Anther color: Near 15A Pollen color: Near 15A

Other Characteristics

Disease resistance.—Neither resistance nor susceptibility observed

What is claimed is:

1. A new and distinct variety of Chrysanthemum plant named ‘MOZA BN 01’ as herein illustrated and described.
